(19) United States
(12) Patent Application Publication
(10) Pub. No.: US 2012/0209119 A1
(43) Pub. Date: Aug. 16, 2012

(54) ULTRASOUND DIAGNOSTIC APPARATUS AND METHOD OF PRODUCING ULTRASOUND IMAGE

(75) Inventor: Yuji OHSHIMA, Kanagawa (JP)

(73) Assignee: FUJIFILM CORPORATION, Tokyo (JP)

(21) Appl. No.: 13/358,547

(22) Filed: Jan. 26, 2012

(30) Foreign Application Priority Data
Feb. 15, 2011 (JP) 2011-029444

(51) Int. Cl. A61B 8/14

(52) U.S. Cl. 600/443

(57) ABSTRACT

An ultrasound diagnostic apparatus includes: a transmission driver which transmits an ultrasonic beam from a transducer array toward a subject; reception signal processors which process reception signals output from the transducer array having received an ultrasonic echo; an image producer which produces image data based on the processed reception signals to produce an ultrasound image; a region detector which detects a region including a high rate change portion, which changes at a rate equal to or higher than a predetermined value, from the ultrasound image as a first measurement region; a controller which controls the transmission driver and the reception signal processors such that transmission and reception of ultrasonic waves are intermittently performed to decrease a frame rate in a second measurement region, where the high rate change portion is not included, less than in the first measurement region.

### BACKGROUND OF THE INVENTION

The present invention relates to an ultrasound diagnostic apparatus and a method of producing an ultrasound image, and in particular, to a technique for suppressing the amount of heat generation in an ultrasound probe of an ultrasound diagnostic apparatus which performs diagnosis on the basis of an ultrasound image produced through transmission and reception of ultrasonic waves with respect to a transducer array of the ultrasound probe.

An ultrasound diagnostic apparatus using an ultrasound image has hitherto been put into practical use in the field of medicine. In general, this type of ultrasound diagnostic apparatus has an ultrasound probe embedded with a transducer array and an apparatus body connected to the ultrasound probe. Ultrasonic waves are transmitted from the ultrasound probe toward a subject, an ultrasonic echo from the subject is received by the ultrasound probe, and the reception signal is electrically processed by the apparatus body to produce an ultrasound image.

In this ultrasound diagnostic apparatus, ultrasonic waves are transmitted from the transducer array, and heat is generated from the transducer array.

On the other hand, since diagnosis is performed while an operator grips the ultrasound probe with one hand and abuts the ultrasound transmission/reception surface of the transducer array on the surface of the subject, there are many cases where the ultrasound probe is accommodated in a small housing such that the operator can easily grip the probe with one hand. For this reason, the temperature in the housing of the ultrasound probe may increase due to heat generation from the transducer array.

In recent years, an ultrasound diagnostic apparatus has been proposed in which a circuit board for a signal process is embedded in the ultrasound probe, and reception signals output from the transducer array are subjected to a digital process and transmitted to the apparatus body through wireless communication or wired communication, thereby reducing the influence of noise to obtain a high image quality ultrasound image.

In the ultrasound probe which performs this type of digital process, heat is generated from the circuit board even when processing the reception signals, and it is necessary to suppress a temperature rise in the housing so as to secure a stable operation of each circuit of the circuit board.

With regard to a countermeasure against a temperature rise of the ultrasound probe, for example, JP 2005-253776 A describes an ultrasound diagnostic apparatus which automatically changes the driving condition of the transducer array depending on the surface temperature of the ultrasound probe. As the surface temperature increases, a driving voltage for each transducer of the transducer array, a number of channels for transmission, a repetition frequency of transmission pulses, a frame rate, and the like when transmitting ultrasonic waves are reduced, such that the surface temperature of the ultrasound probe is maintained at an appropriate temperature.

However, in the apparatus of JP 2005-253776 A which changes the driving condition of the transducer array at the time of transmission, it is difficult to cope with heat generation at the time of reception in the ultrasound probe which performs the above-described digital process.

### SUMMARY OF THE INVENTION

The invention has been finalized in order to solve the problems in the related art, and an object of the invention is to provide an ultrasound diagnostic apparatus and a method of producing an ultrasound image capable of obtaining a high image quality ultrasound image while suppressing a rise in the internal temperature of an ultrasound probe.

An ultrasound diagnostic apparatus according to the present invention comprises:
a transducer array;
a transmission driver which transmits an ultrasonic beam from the transducer array toward a subject;
reception signal processors which process reception signals output from the transducer array having received an ultrasonic echo from the subject;
an image producer which produces image data based on the reception signals processed by the reception signal processors to produce an ultrasound image;
a region detector which detects a region including a high rate change portion, which changes at a rate equal to or higher than a predetermined value, from the ultrasound image as a first measurement region;
a controller which controls the transmission driver and the reception signal processors such that transmission and reception of ultrasonic waves from the transducer array are intermittently performed to decrease a frame rate in a second measurement region, where the high rate change portion is not included, less than in the first measurement region detected by the region detector.

A method of producing an ultrasound image according to the present invention comprises the steps of:
transmitting an ultrasonic beam from a transducer array toward a subject on the basis of driving signals supplied from a transmission driver;
processing reception signals output from the transducer array having received an ultrasonic echo from the subject by reception signal processors;
producing an ultrasound image on the basis of the reception signals processed by the reception signal processor;
detecting a region including a high rate change portion, which changes at a rate equal to or higher than a predetermined value, from the ultrasound image as a first measurement region; and
controlling the transmission driver and the reception signal processors such that transmission and reception of ultrasonic waves from the transducer array are intermittently performed so as to decrease a frame rate in a second measurement region, where the high rate change portion is not included, less than in the detected first measurement region.

### BRIEF DESCRIPTION OF THE DRAWINGS

FIG. 1 is a block diagram showing the configuration of an ultrasound diagnostic apparatus according to Embodiment 1 of the invention.

FIG. 2 is a diagram showing sequentially produced frames of an ultrasound image produced in a high image quality mode.

FIG. 3 is a diagram showing sequentially produced frames of an ultrasound image produced in a temperature rise suppression mode.

FIG. 4 is a diagram showing the ultrasound image of a condition in which a mode is changed between a high image quality mode and a temperature rise suppression mode.

FIG. 5 is a diagram showing sequentially produced frames of an ultrasound image produced in a temperature rise suppression mode in Embodiment 2.

FIG. 6 is a diagram showing sequentially produced frames of an ultrasound image produced in a temperature rise suppression mode in Embodiment 3.

### DETAILED DESCRIPTION OF THE INVENTION

Hereinafter, embodiments of the invention will be described with reference to the accompanying drawings.

### Embodiment 1

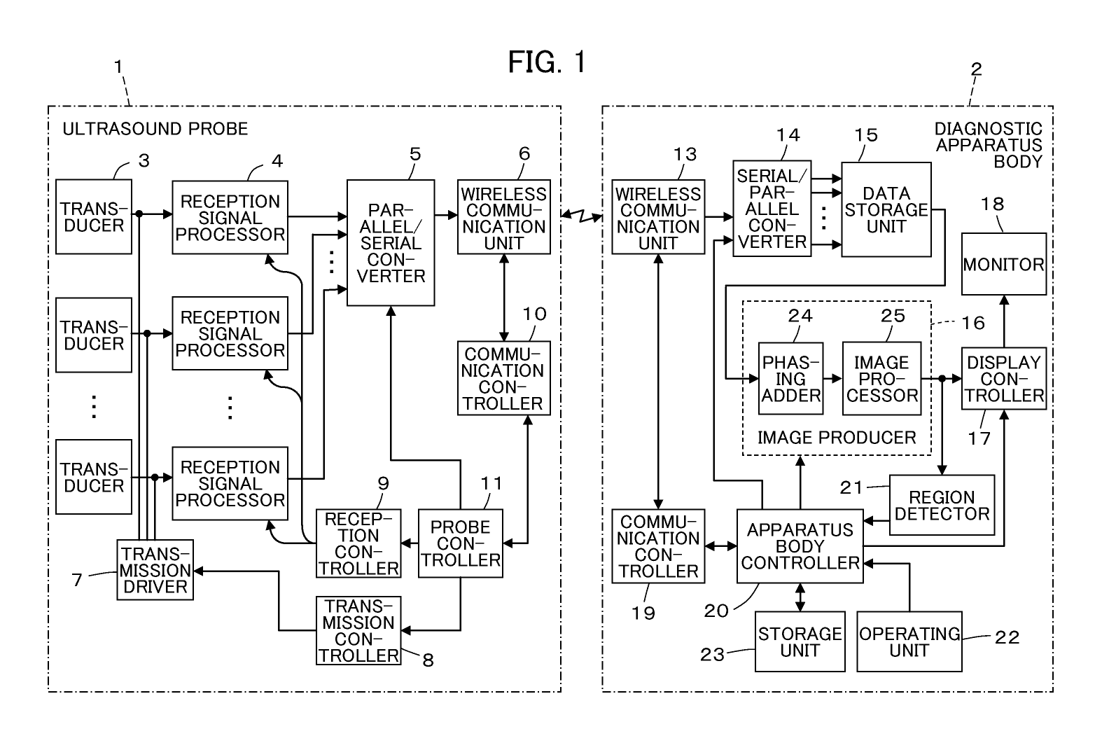

FIG. 1 shows the configuration of an ultrasound diagnostic apparatus according to Embodiment 1 of the invention. The ultrasound diagnostic apparatus includes an ultrasound probe 1, and a diagnostic apparatus body 2 connected to the ultrasound probe 1 through wireless communication.

The ultrasound probe 1 has a plurality of ultrasound transducers 3 constituting a plurality of channels of a one-dimensional or two-dimensional transducer array. Reception signal processors 4 are correspondingly connected to the transducers 3, and a wireless communication unit 6 is connected to the reception signal processors 4 through a parallel/serial converter 5. A transmission controller 8 is connected to a plurality of transducers 3 through the transmission driver 7, a reception controller 9 is connected to the reception signal processors 4, and a communication controller 10 is connected to the wireless communication unit 6. A probe controller 11 is connected to the parallel/serial converter 5, the transmission controller 8, the reception controller 9, and the communication controller 10.

The plurality of transducers 3 transmit ultrasonic waves in response to driving signals supplied from the transmission driver 7, receive an ultrasonic echo from the subject, and output reception signals. Each transducer 3 is constituted by a vibrator in which electrodes are formed at both ends of a piezoelectric body made of piezoelectric ceramic represented by PZT (Pb (lead) zirconate titanate), a polymer piezoelectric device represented by PVDF (polyvinylidene difluoride), piezoelectric single crystal represented by PMN-PT (lead magnesium niobate-lead titanate solid solution), or the like.

If a pulsed or continuous-wave voltage is applied across the electrodes of the vibrator, the piezoelectric body expands and contracts. Pulsed or continuous-wave ultrasonic waves are produced from the vibrators and synthesized to form an ultrasonic beam. When receiving the propagating ultrasonic waves, the vibrators expand and contract to produce electric signals, and the electric signals are output as the reception signals of the ultrasonic waves.

The transmission driver 7 includes, for example, a plurality of pulsars. The transmission driver 7 adjusts the delay amount of each of the driving signals on the basis of a transmission delay pattern selected by the transmission controller 8 such that ultrasonic waves transmitted from the transducers 3 form a wide ultrasonic beam which covers an area of a tissue of the subject, and supplies the driving signals to the transducers 3.

The reception signal processor 4 of each channel performs an orthogonal detection process or an orthogonal sampling process on the reception signal output from the corresponding transducer 3 under the control of the reception controller 9 to produce a complex baseband signal, samples the complex baseband signal to produce sample data including information of the area of the tissue, and supplies sample data to the parallel/serial converter 5. The reception signal processor 4 may perform a data compression process for high-efficiency encoding on data obtained by sampling the complex baseband signal to produce sample data.

The parallel/serial converter 5 converts parallel sample data produced by the reception signal processors 4 of a plurality of channels to serial sample data.

The wireless communication unit 6 modulates a carrier on the basis of serial sample data to produce a transmission signal and transmits the transmission signal to an antenna to transmit a radio wave from the antenna, thereby transmitting serial sample data. As a modulation system, for example, ASK (Amplitude Shift Keying), PSK (Phase Shift Keying), QPSK (Quadrature Phase Shift Keying), 16QAM (16 Quadrature Amplitude Modulation), or the like is used.

The wireless communication unit 6 performs wireless communication with the diagnostic apparatus body 2 to transmit sample data to the diagnostic apparatus body 2. Simultaneously, the wireless communication unit 6 receives various control signals from the diagnostic apparatus body 2, and outputs the received control signals to the communication controller 10. The communication controller 10 controls the wireless communication unit 6 such that sample data is transmitted with transmission radio field intensity set by the probe controller 11, and outputs various control signals received by the wireless communication unit 6 to the probe controller 11.

The probe controller 11 controls the operations of the respective units of the ultrasound probe 1 on the basis of various control signals transmitted from the diagnostic apparatus body 2.

The ultrasound probe 1 is embedded with a battery (not shown), and power is supplied from the battery to each circuit in the ultrasound probe 1.

The ultrasound probe 1 may be an external probe, such as a linear scan type, a convex scan type, or a sector scan type, or may be a probe for an ultrasound endoscope, such as a radial scan type.

The diagnostic apparatus body 2 has a wireless communication unit 13. A data storage unit 15 is connected to the wireless communication unit 13 through a serial/parallel converter 14, and an image producer 16 is connected to the data storage unit 15. A monitor 18 is connected to the image producer 16 through a display controller 17. A communication controller 19 is connected to the wireless communication unit 13, and an apparatus body controller 20 is connected to the serial/parallel converter 14, the image producer 16, the display controller 17, and the communication controller 19. A region detector 21 is connected to the image producer 16, and the region detector 21 is connected to the apparatus body controller 20. An operating unit 22 which is used when the operator performs an input operation and a storage unit 23 which stores an operation program are connected to the apparatus body controller 20.

The wireless communication unit 13 performs wireless communication with the ultrasound probe 1 to transmit various control signals to the ultrasound probe 1. The wireless communication unit 13 demodulates a signal received by the antenna to output serial sample data.

The communication controller 19 controls the wireless communication unit 13 such that various control signals are transmitted with transmission radio field intensity set by the apparatus body controller 20.

The serial/parallel converter 14 converts serial sample data output from the wireless communication unit 13 to parallel sample data. The data storage unit 15 is constituted by a memory, a hard disk, or the like, and stores sample data for at least one frame converted by the serial/parallel converter 14.

The image producer 16 performs a reception focus process on sample data for every frame read from the data storage unit 15 to produce an image signal representing an ultrasound diagnostic image. The image producer 16 includes a phasing adder 24 and an image processor 25.

The phasing adder 24 performs a reception focus process by selecting one reception delay pattern from among a plurality of reception delay patterns stored in advance in accordance with the reception direction set in the apparatus body controller 20, giving the delay to each of a plurality of complex baseband signals represented by sample data on the basis of the selected reception delay pattern, and adding the complex baseband signals. With this reception focus process, the focus of the ultrasonic echo is narrowed to produce a baseband signal (sound ray signal).

The image processor 25 produces a B-mode image signal, which is tomographic image information relating to the tissue of the subject, on the basis of the sound ray signal produced by the phasing adder 24. The image processor 25 includes an STC (sensitivity time control) unit, an interpolator, and a DSC (digital scan converter). The STC unit corrects attenuation depending on the distance in accordance with the depth of the reflection position of the ultrasonic wave for the sound ray signal. The interpolator performs an interpolation process on a missing frame of the sound ray signal by intermittent transmission and reception of ultrasonic waves in a temperature rise suppression mode described below. The DSC converts (raster-converts) the sound ray signal corrected by the STC unit to an image signal based on a normal television signal scan system, and performs a necessary image process, such as a gradation process, to produce a B-mode image signal.

The display controller 17 displays an ultrasound diagnostic image on the monitor 18 on the basis of an image signal produced by the image producer 16. The monitor 18 includes, for example, a display device, such as an LCD, and displays an ultrasound diagnostic image under the control of the display controller 17.

The region detector 21 detects a region including a high rate change portion, which changes at a rate equal to or higher than a predetermined value, as a first measurement region and the remaining region as a second measurement region from the ultrasound image produced by the image producer 16. For example, a binarization process or the like is performed on each portion (an internal organ of a human body, or the like) in the ultrasound image to extract the contour of the corresponding portion, and the rate at which each portion changes by a change amount of the contour of each portion for each frame is obtained. Subsequently, the range which is covered by a scan line group passing through a portion (high rate change portion) which changes at a rate equal to or higher than a predetermined value is detected as the first measurement region, and the remaining range is detected as the second measurement region.

When the entire region of the ultrasound image is detected by the region detector 21 as the first measurement region, the apparatus body controller 20 performs control such that the reception signal processor 4 and the transmission driver 7 of the ultrasound probe 1 are operated in a high image quality mode. When the second measurement region including no high rate change portion is detected by the region detector 21, the apparatus body controller 20 performs control such that the reception signal processor 4 and the transmission driver 7 of the ultrasound probe 1 are operated in a temperature rise suppression mode.

In this diagnostic apparatus body 2, the serial/parallel converter 14, the image producer 16, the display controller 17, the communication controller 19, and the apparatus body controller 20 are constituted by a CPU and an operation program which causes the CPU to perform various processes, and these may be constituted by digital circuits. The operation program is stored in the storage unit 23. As a recording medium in the storage unit 23, in addition to an internal hard disk, a flexible disk, an MO, an MT, a RAM, a CD-ROM, a DVD-ROM, an SD card, a CF card, a USB memory, or a server, or the like may be used.

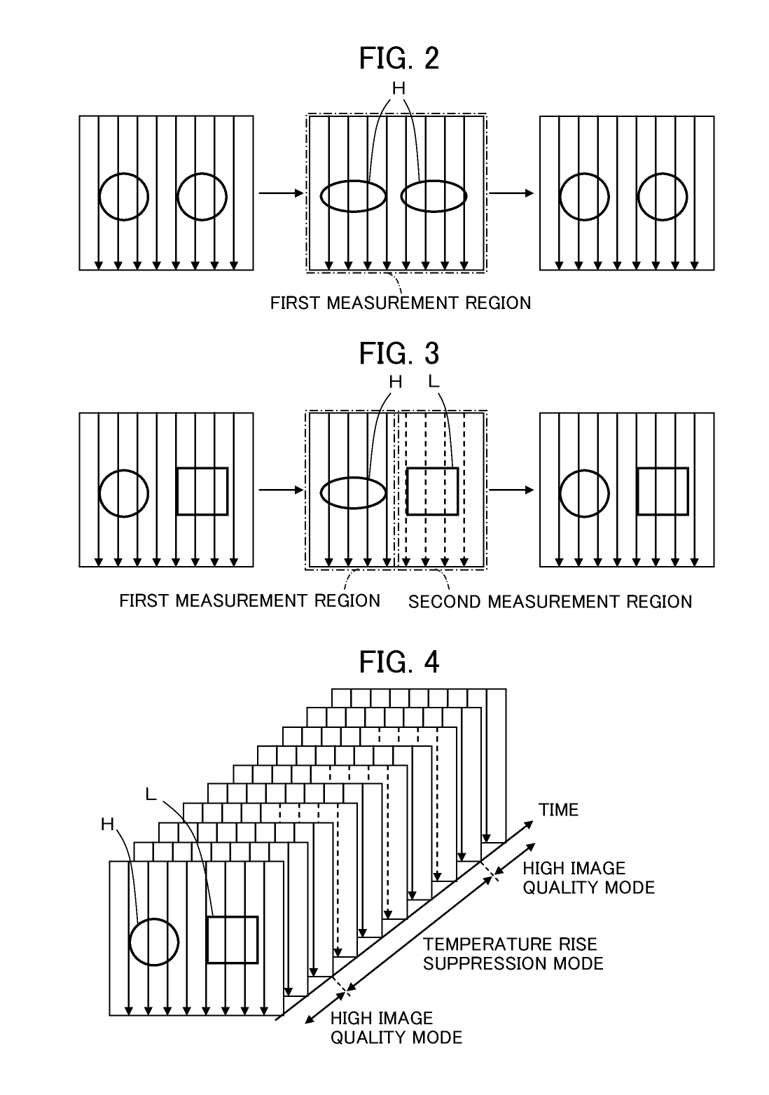

The high image quality mode and the temperature rise suppression mode will be described with reference to FIGS. 2 and 3.

A frame rate necessary for capturing a change differs between a high rate change portion H which changes at a rate equal to or higher than a predetermined value and a low rate change portion L which changes at a rate lower than the predetermined value. That is, with regard to the low rate change portion L, a change can be captured at a frame rate lower than the high rate change portion H. Accordingly, with regard to the first measurement region which is covered by a scan line group passing through the high rate change portion H, a high frame rate is maintained, and with regard to the second measurement region other than the first measurement region, the frame rate can be lowered.

For example, as shown in FIG. 2, the high image quality mode is set when only the first measurement region is detected in each frame of an ultrasound image to be sequentially produced. In this case, the transmission driver 7 is controlled by the transmission controller 8 and the reception signal processor 4 is controlled by the reception controller 9 such that the plurality of transducers 3 constituting the transducer array are continuously operated with respect to all scan lines over the entire region of the ultrasound image. In this case, it is possible to obtain a high-quality image by continuously transmitting and receiving with respect to the first measurement region over the entire region of the ultrasound image.

As shown in FIG. 3, the temperature rise suppression mode is set when the second measurement region other than the first measurement region is detected in each frame of an ultrasound image to be sequentially produced. As in the high image quality mode, the transmission driver 7 is controlled by the transmission controller 8 and the reception signal processor 4 is controlled by the reception controller 9 such that the transducers 3 are continuously operated in the first measurement region and the transducers 3 are alternately operated and stopped for each frame in the second measurement region. In this way, even when ultrasonic waves are intermittently transmitted and received with respect to the second measurement region, it is possible to capture a change in the low rate change portion L, and since the transmission driver 7 or the reception signal processor 4 is stopped at a given interval, it is possible to suppress a rise in the internal temperature of the ultrasound probe 1.

With the transmission and reception of ultrasonic waves in the temperature rise suppression mode, the frame rate of the second measurement region is lowered compared to the first measurement region, when producing an ultrasound image, a frame where sound ray signals of the second measurement region are missing occurs. With regard to the missing frame, the interpolator of the image producer 16 performs interpolation using the sound ray signals of the second measurement region produced in the previous and subsequent frames.

Next, the operation of Embodiment 1 will be described.

If ultrasound diagnosis starts, first, the apparatus body controller 20 selects the high image quality mode. The apparatus body controller 20 controls the transmission driver 7 and the reception signal processor 4 through the probe controller 11 of the ultrasound probe 1. Ultrasonic waves are transmitted from the plurality of transducers 3 to all the scan lines in response to driving signals supplied from the transmission driver 7. A reception signal output from each transducer 3 having received an ultrasonic echo from the subject is supplied to the corresponding reception signal processor 4. The reception signals supplied to the reception signal processor 4 are sequentially converted to sample data. Sample data is converted to serial sample data by the parallel/serial converter 5, and serial sample data is transmitted from the wireless communication unit 6 to the diagnostic apparatus body 2 in a wireless manner. Sample data received by the wireless communication unit 13 of the diagnostic apparatus body 2 is converted to parallel data by the serial/parallel converter 14, and parallel data is stored in the data storage unit 15. Sample data for each frame is read from the data storage unit 15, an image signal is produced by the image producer 16, and an ultrasound diagnostic image is displayed on the monitor 18 on the basis of the image signal by the display controller 17.

In the ultrasound image in the high image quality mode produced by the image producer 16 in the above-described manner, the detection of the first measurement region is attempted by the region detector 21. The region detector 21 obtains a deformation amount of each portion (an internal organ of a human body, or the like) in the ultrasound image for each frame, detects the range, which is covered by a scan line group passing through the high rate change portion H having a deformation rate equal to or higher than a predetermined value, as the first measurement region, and detects the remaining range as the second measurement region. The detection result by the region detector 21 is input to the apparatus body controller 20.

Subsequently, when the detection result of the region detector 21 shows that only the first measurement region is detected, the apparatus body controller 20 maintains a high frame rate in the high image quality mode over the entire region. When the second measurement region other than the first measurement region is detected, the apparatus body controller 20 is changed to the temperature rise suppression mode.

For example, as shown in FIG. 4, the ultrasound image in the high image quality mode including the high rate change portion H and the low rate change portion L is produced, and when the first measurement region including the high rate change portion H and the remaining second measurement region are detected by the region detector 21, the apparatus body controller 20 selects the temperature rise suppression mode, and controls the transmission driver 7 and the reception signal processors 4 through the probe controller 11 of the ultrasound probe 1 such that transmission and reception of ultrasonic waves are continuously performed in the first measurement region, and transmission and reception of ultrasonic waves are intermittently performed in the second measurement region.

As described above, even when an ultrasound image includes a plurality of portions which are different in the change rate, the frame rate is adjusted depending on the change rate of each portion, thereby suppressing a rise in the temperature of the ultrasound probe 1 while minimizing deterioration of image quality.

Similarly, a reception signal obtained in the temperature rise suppression mode is transmitted from the ultrasound probe 1 to the diagnostic apparatus body 2 in a wireless manner, supplied to the image producer 16 of the diagnostic apparatus body 2, and converted to an image signal. At this time, ultrasonic waves are intermittently transmitted and received with respect to the second measurement region, such that a frame where the image signal of the second measurement region is missing is sequentially interpolated by the interpolator of the image producer 16 using the image signals of the second measurement region produced in the previous and subsequent frames.

An ultrasound diagnostic image is displayed on the monitor 18 on the basis of the image signal produced in the above-described manner by the display controller 17.

Subsequently, if transmission and reception of ultrasonic waves in the temperature rise suppression mode have been performed for a given time, as shown in FIG. 4, the high image quality mode is selected again by the apparatus body controller 20, and the detection of the first measurement region and the second measurement region from the ultrasound image in the high image quality mode is performed by the region detector 21.

As described above, it is possible to change the number of transmissions/receptions of ultrasonic waves from the transducers 3 for each portion depending on the change rate of each portion in the ultrasound image. The reduction in the number of transmissions/receptions of ultrasonic waves allows the interpolration of missing image data of the second measurement region using image data obtained previously and subsequently. For this reason, it becomes possible to suppress a rise in the temperature of the ultrasound probe 1 without deteriorating image quality necessary for diagnosis, that is, without affecting diagnosis.

Although in this embodiment, the change from the temperature rise suppression mode to the high image quality mode is done when the control in the temperature rise suppression mode has been performed for a given time, the invention is not limited thereto. For example, the change from the temperature rise suppression mode to the high image quality mode may be done when the shift of a site under ultrasound diagnosis to another site is detected by detecting the movement of the ultrasound probe 1.

### Embodiment 2

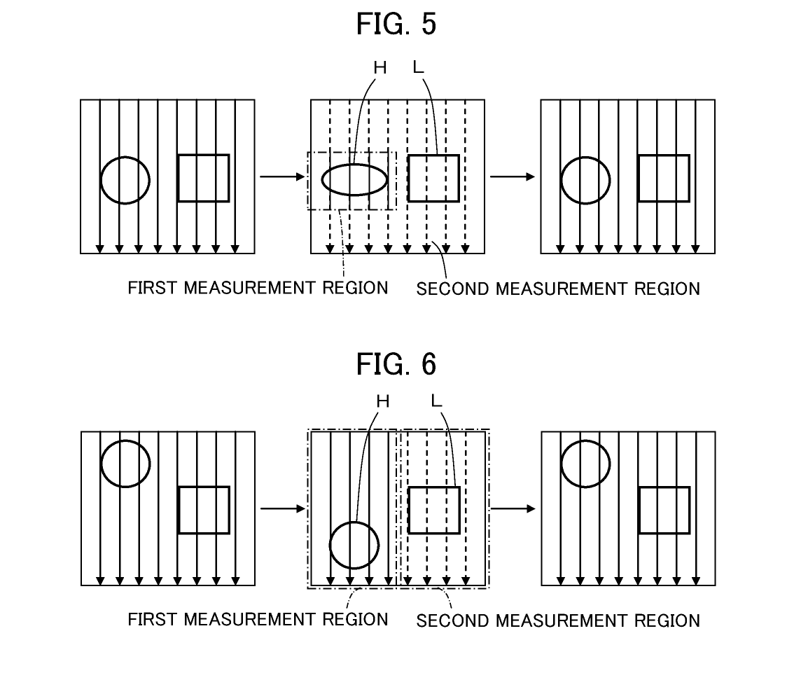

Although in Embodiment 1, the range which is covered by a scan line group passing through the high rate change portion H is defined as the first measurement region, the invention is not limited thereto. As shown in FIG. 5, a region covered by a scan line group passing through the high rate change portion H and corresponding to a depth position of the high rate change portion H may be defined as the first measurement region.

If the high image quality mode is selected by the apparatus body controller 20 and an ultrasound image in the high image quality mode is produced, the region detector 21 detects a region covered by a scan line group passing through the high rate change portion H and corresponding to a depth position of the high rate change portion H as the first measurement region, and detects the remaining region as the second measurement region. When the detection result of the region detector 21 shows that only the first measurement region is detected, the apparatus body controller 20 selects the high image quality mode. When the second measurement region other than the first measurement region is detected, the apparatus body controller 20 selects the temperature rise suppression mode.

In the temperature rise suppression mode, as in Embodiment 1, transmission and reception of ultrasonic waves are intermittently performed with respect to the scan lines of the second measurement region not passing through the high rate change portion H. Transmission of ultrasonic waves is continuously performed with respect to the scan lines of the first measurement region and the second measurement region passing through the high rate change portion H, and only an ultrasonic echo from the first measurement region is continuously received. That is, with regard to continuously transmitted ultrasonic waves, the reception signal processors 4 are continuously in the ON state for the time corresponding to the measurement depth of the first measurement region, and reception of an ultrasonic echo is continuously performed. The reception signal processors 4 are in the OFF state at a predetermined interval for the time corresponding to the measurement depth of the second measurement region, and reception of an ultrasonic echo is intermittently performed.

An image signal in the temperature rise suppression mode obtained in the above-described manner is missing at a predetermined interval in the second measurement region due to intermittent transmission and reception of ultrasonic waves. A frame where an image signal is missing is sequentially interpolated by the interpolator of the image producer 16 using image signals of the second measurement region in the previous and subsequent frames.

Accordingly, as shown in FIG. 5, processing of a reception signal is performed by the reception signal processors 4 only for the time necessary for receiving an ultrasonic echo from a high rate change region, and is not performed for the remaining time. Therefore, it becomes possible to suppress a rise in the temperature of the ultrasound probe 1 while minimizing deterioration in image quality.

### Embodiment 3

Although in Embodiments 1 and 2, a portion which is deformed at equal to or higher than a predetermined rate is defined as a high rate change portion, the invention is not limited thereto. As shown in FIG. 6, a portion which moves at a rate equal to or higher than a predetermined value may be defined as the high rate change portion H.

If the high image quality mode is selected by the apparatus body controller 20, and the ultrasound image of the high image quality mode is produced, the region detector 21 detects the range, which is covered by a scan line group passing through the high rate change portion H moving at a rate equal to or higher than a predetermined value, as the first measurement region, and detects the remaining region as the second measurement region from the ultrasound image. When the detection result of the region detector 21 shows that only the first measurement region is detected, the apparatus body controller 20 selects the high image quality mode. When the second measurement region other than the first measurement region is detected, the apparatus body controller 20 selects the temperature rise suppression mode.

In the temperature rise suppression mode, the apparatus body controller 20 controls the transmission driver 7 and the reception signal processors 4 through the probe controller 11 of the ultrasound probe 1 such that transmission and reception of ultrasonic waves are continuously performed with respect to the first measurement region, and transmission and reception of ultrasonic waves are intermittently performed with respect to the second measurement region.

As described above, the number of transmissions/receptions of ultrasonic waves from the transducers 3 for each portion changes depending on the movement rate of each portion in the ultrasound image, thereby suppressing a rise in the temperature of the ultrasound probe 1 while minimizing deterioration of image quality.

What is claimed is:

1. An ultrasound diagnostic apparatus comprising:
a transducer array;
a transmission driver which transmits an ultrasonic beam from the transducer array toward a subject;
reception signal processors which process reception signals output from the transducer array having received an ultrasonic echo from the subject;
an image producer which produces image data based on the reception signals processed by the reception signal processors to produce an ultrasound image;
a region detector which detects a region including a high rate change portion, which changes at a rate equal to or higher than a predetermined value, from the ultrasound image as a first measurement region;
a controller which controls the transmission driver and the reception signal processors such that transmission and reception of ultrasonic waves from the transducer array are intermittently performed to decrease a frame rate in a second measurement region, where the high rate change portion is not included, less than in the first measurement region detected by the region detector.

2. The ultrasound diagnostic apparatus according to claim 1,
wherein the region detector detects a region including the high rate change portion, in which at least one of a deformation rate and a movement rate in the ultrasound image is equal to or higher than a predetermined value, as the first measurement region.

3. The ultrasound diagnostic apparatus according to claim 1,
wherein the region detector detects a range covered by a scan line group passing through the high rate change portion as the first measurement region.

4. The ultrasound diagnostic apparatus according to claim 3,
wherein the region detector detects a region corresponding to a deep position of the high rate change portion as the first measurement region.

5. The ultrasound diagnostic apparatus according to claim 1,
wherein the region detector repeatedly detects the first measurement region for every given time.

6. The ultrasound diagnostic apparatus according to claim 1,
wherein a frame in which image data of the second measurement region is missing with the decrease in the frame rate is interpolated using image data of the second measurement region produced in the previous and subsequent frames.

7. A method of producing an ultrasound image, the method comprising the steps of:
transmitting an ultrasonic beam from a transducer array toward a subject on the basis of driving signals supplied from a transmission driver;
processing reception signals output from the transducer array having received an ultrasonic echo from the subject by reception signal processors;
producing an ultrasound image on the basis of the reception signals processed by the reception signal processor;
detecting a region including a high rate change portion, which changes at a rate equal to or higher than a predetermined value, from the ultrasound image as a first measurement region; and
controlling the transmission driver and the reception signal processors such that transmission and reception of ultrasonic waves from the transducer array are intermittently performed so as to decrease a frame rate in a second measurement region, where the high rate change portion is not included, less than in the detected first measurement region.
